(19) United States
(12) Patent Application Publication
(10) Pub. No.: US 2009/0024015 A1
(43) Pub. Date: Jan. 22, 2009

(54) SENSING ELEMENT HAVING AN ADHESIVE BACKING

(75) Inventor: Kenneth M. Curry, Oceanside, CA (US)

Correspondence Address: EDWARDS LIFESCIENCES CORPORATION, LEGAL DEPARTMENT, ONE EDWARDS WAY, IRVINE, CA, 92614, US

(73) Assignee: Edwards Lifesciences Corporation, Irvine, CA (US)

(21) Appl. No.: 12/167,903

(22) Filed: Jul. 3, 2008

Related U.S. Application Data
(60) Provisional application No. 60/950,317, filed on Jul. 17, 2007.

(51) Int. Cl. A61B 5/055; B32B 37/12

(52) U.S. Cl. 600/347; 156/60

(57) ABSTRACT

The present invention is directed to a sensing element that comprises a flexible substrate having first and second opposite surfaces; at least one sensor disposed on the first surface of the flexible substrate; an adhesive layer substantially covering the second surface of the flexible substrate; and a release liner releasably adhered to the adhesive layer so that upon removal of the release liner the adhesive layer is exposed for securing the sensing element to the catheter. The release liner permits the sensing element to be positioned at a desired location within the catheter after which the release liner can be removed to expose the adhesive layer. The adhesive layer can then be used to attach and secure the sensing element at a desired location on the catheter. As a result, the need for additional adhesives can be reduced or eliminated.

### CLAIM OF PRIORITY UNDER 35 U.S.C. §119

The present Application for Patent claims priority to Provisional Application No. 60/950,317 filed Jul. 17, 2007, and assigned to the assignee hereof and hereby expressly incorporated by reference herein.

### FIELD OF THE INVENTION

The present invention relates generally to catheters for use in medical applications and more particularly to catheters having an integral sensing element.

### BACKGROUND OF THE INVENTION

It is common in many medical procedures to monitor vital signs and other biomedical or physiological parameters of a patient. This is particularly true of patients in intensive care units (ICUs) or other emergency situations where accurate and timely monitoring of such parameters can be the difference between life and death. In such situations, the practice of drawing a blood sample for laboratory analysis may be too slow.

To provide a more timely method of monitoring blood chemistry, various sensing elements have been developed in which a sensor is placed into a patient's bloodstream to provide real-time monitoring of one or more physiological parameters. For example, amperometric biosensors have been developed in which the concentration of an analyte in a patient's bloodstream can be determined by positioning, within the circulatory system, a chemical electrode that produces an electrical current proportional to the concentration of the analyte. Such sensors can be used to monitor physiological parameters continuously over many hours, or perhaps even days, using analytical electronics coupled to the sensor through a conductive interface.

One common method of using a sensor includes the use of single or multiple lumen catheters in which a sensing element having a sensor is positioned towards the distal end of the catheter. Generally, the sensor is positioned within the catheter so that its circuitry is shielded from the direct flow of the patient's blood. A medical grade adhesive, such as an epoxy, is typically used to secure the sensor to an inner wall of the catheter. In order for the catheter and sensor to be suspended within a patient's blood vessel, it is important for the catheter and sensor to have a relatively small size, while still having sufficient mechanical integrity to withstand the rigors of installation. However, manipulating and properly positioning the sensing element in the catheter can present challenges. For example, in some cases the application of the adhesive to the sensor can result in the depositing of adhesive in undesirable locations on the sensor, such as on the chemical electrode or a membrane that can be associated with the electrode. This can result in the sensor being damaged or unusable.

Thus, there still exists a need for an improved catheter assembling and a method of securing and positioning a sensor within a catheter.

### BRIEF SUMMARY OF THE INVENTION

The present invention is directed to a sensing element and a method of positioning and securing the sensing element within a catheter that overcomes many of the problems associated with the prior art. In one embodiment, the sensing element comprises a flexible substrate having first and second opposite surfaces; at least one sensor disposed on the first surface of the flexible substrate; an adhesive layer covering at least a portion of and preferably a substantial portion of the second surface of the flexible substrate; and a release liner releasably adhered to the adhesive layer so that upon removal of the release liner the adhesive layer is exposed for securing the sensing element to the catheter. The release liner permits the sensing element to be positioned at a desired location within the catheter after which the release liner can be removed to expose the adhesive layer. The adhesive layer can then be used to attach and secure the sensing element at a desired location on the catheter. As a result, the need for additional adhesives can be eliminated.

The sensing element having the adhesive layer and release liner can be used in conjunction with a variety of different catheters, such as a single lumen catheter, a multilumen catheter, a central venous catheter (CVC), a pulmonary artery catheter (PAC), or a peripherally inserted central catheter (PICC). In one embodiment, the sensing element can be used in combination with a catheter assembly having an elongated tube that includes a recess formed in an outer wall of the catheter having a surface for receiving the sensing element. The recess typically communicates with a lumen of the catheter. In one particular embodiment, the recess comprises an opening formed in an outer wall of the catheter defining a sensing port in which the sensing element can be positioned. In some embodiments, the sensing element can be disposed within a lumen disposed within the tube of the catheter. In other embodiments, the sensing element can be disposed in the recess adjacent to an outer wall of the catheter.

A catheter assembly in accordance with the present invention can be assembled by introducing and positioning the sensing element in a recess formed in an outer wall of the catheter. In one embodiment, the sensing element can be introduced into a lumen of the catheter through a sensing port. The presence of the release liner permits the manipulation of the sensing element within the catheter or lumen without adversely effecting or disturbing the adhesive layer. As a result, the sensing element can be positioned in or adjacent to a desired location on the catheter or within the lumen prior to removing the release liner. After the sensing element is positioned approximate or near a desired location, the release liner can be removed by pulling it away from the adhesive layer, to thereby expose the adhesive layer. The sensing element can then be attached to the catheter by contacting the exposed adhesive layer to a surface of the catheter. Preferably, the adhesive layer comprises a pressure sensitive adhesive that permits the sensing element to be securely anchored to the catheter by applying manual pressure against the sensing element in the direction of a surface of the catheter. In one embodiment, the sensing element is adhered to inner wall of a lumen of the catheter. In another embodiment, the sensing element is positioned in the recess so that it is flush with an outer surface of the catheter.

The sensing element can include a wide variety of different sensors such as an optical fiber, a pH sensor, a pressure sensor, a pacing electrode, at least one pacing lead, one or more electrodes for glucose monitoring, and combinations thereof. In one embodiment, the sensor can comprise one or more electrodes mounted on the flexible substrate, such as a working electrode, a counter electrode, and a reference electrode. For example, in one embodiment the sensor can comprise an enzyme-based biosensor in which an analyte-concentration-dependent biochemical reaction signal is converted into a measurable physical signal, such as an optical or electrical signal. Such biosensors can be used to measure the concentration of a wide variety of analytes such as glucose, lactate, cholesterol, bilirubin and amino acids. In one particular embodiment, the sensing element can include a glucose sensor in which an electrode is at least partially coated with a glucose oxidase enzyme for monitoring glucose levels in the blood stream of a patient.

From the foregoing and following discussion, it should be evident to the reader that the present invention overcomes many of the problems and disadvantages associated with the prior art.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1A is an illustration of a sensing element in the form of a flex circuit having a sensor on one surface and an adhesive layer disposed on an opposite surface that is protected with a release liner;

FIG. 1B is a magnified cross-sectional side view of the sensing element of FIG. 1A taken along line 1B of FIG. of 1A;

FIG. 2 is a side view of a multilumen catheter assembly according to an embodiment of the invention;

FIG. 3 is a magnified detail of the distal end of the multilumen catheter of FIG. 2 according to an embodiment of the invention; and

FIGS. 4A through 4C depict in a step-wise manner a method of attaching a sensing element to the inner wall of a lumen of a catheter that is in accordance with one embodiment of the invention;

FIG. 4D illustrates an embodiment of the invention in which the sensing element is disposed in a recess formed in an outer wall of the catheter; and

FIG. 5 is perspective view of a flexible structure having a plurality of electrodes disposed on a surface thereof and an adhesive layer with a protecting release liner disposed on an opposite surface.

### DETAILED DESCRIPTION OF THE INVENTION

The present invention now will be described more fully hereinafter with reference to the accompanying drawings, in which some, but not all embodiments of the invention are shown. Indeed, the invention can be embodied in many different forms and should not be construed as limited to the embodiments set forth herein; rather, these embodiments are provided so that this disclosure will satisfy applicable legal requirements. Like numbers refer to like elements throughout.

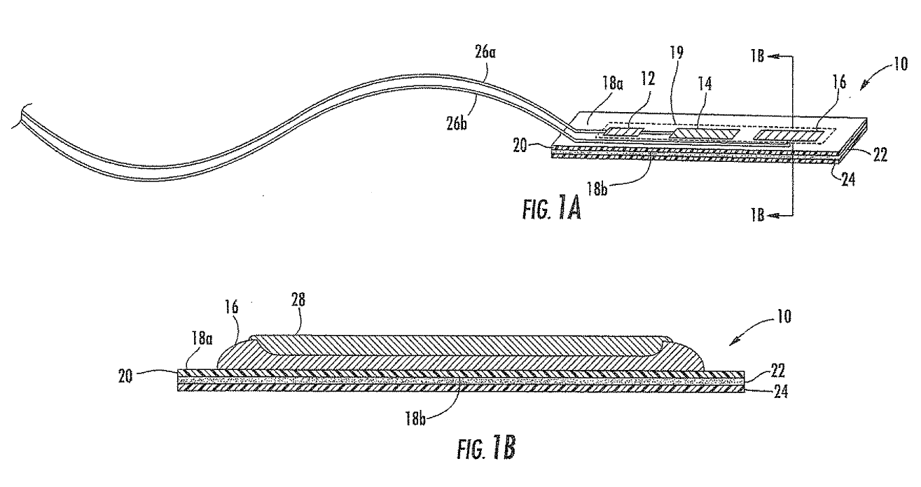

The present invention provides a catheter assembly and associated method for securing and positioning a sensing element within a catheter that eliminates the need for applying an additional adhesive to the sensing element at the time the sensing element is being positioned within the catheter With reference to FIG. 1, an exemplary sensing element having at least one sensor that is in accordance with one embodiment of the invention is illustrated and broadly designated by reference number 10. The sensing element 10 comprises a flexible substrate 20 having opposite first and second surfaces 18a, 18b, respectively, and including one or more sensors 19 disposed on at least one surface 18a thereof. As discussed in greater detail below, the sensor is capable of measuring one or more physiological parameters. In the illustrated embodiment, the sensor 19 (represented by the dashed lines) comprises a plurality of electrodes 12, 14, 16 that are used to measure one or more physiological parameters. An adhesive layer 22 is applied to the surface 18b of the flexible substrate 20 that is opposite surface 18a. The adhesive layer covers at least a portion of the surface 18b of the flexible substrate so that there is sufficient adhesion to securely attach the sensing element to a catheter. Preferably, the adhesive layer 22 covers a substantial portion of the surface 18b (e.g. greater than 50%, greater than 75% or even greater than 90%). A release liner 24 substantially covers the adhesive layer to thereby protect the adhesive from premature contact with objects or other portions of the catheter assembly. The release liner 24 is releasably adhered to the adhesive layer 22 and protects the adhesive before use. At a desired time, the release liner 24 can be removed to expose the adhesive layer 22. The adhesive layer 22 can then be used to secure the sensing element 10 to the catheter at a desired location, e.g., within the outer wall of the catheter or within a lumen of the catheter. The use of the previously applied adhesive layer 22 and release liner 24 helps to permit the quick and efficient assembly and positioning of the sensing element 10 within the catheter while helping eliminate the need for applying an additional adhesive to the sensing element just prior to securing the sensing element to the catheter.

The sensing element 10 can also include electrical wires 26a, 26b for communicating and transmitting power to the sensor, e.g., for sustaining an oxidation or reduction reaction, and can also carry signal currents to a detection circuit (not shown) indicative of a physiological parameter being measured. In one embodiment, the electrical wires 26a, 26b can be coupled or soldered to conductive traces formed on the substrate 20 using flex circuit technology. For example, the traces can be gold-plated copper. In one embodiment, the sensing element 10 can be designed so that the flex circuit terminates to a tab that mates to a multi-pin connector, such as a 3-pin, 1 mm pitch ZIF Molex connector. Such a connection can help facilitate excitation of one or more of the electrodes and measurement of electrical current signals, for example, using a potentiostat or other controller. This embodiment is also advantageous in that the elongated flex circuit can help eliminate the need for separate wires, attachments, and encapsulation.

The flexible substrate 20 is typically formed of a relatively thin polymeric film that can be used to dispose one or more electrodes or flexible circuits thereon. For example, the flexible substrate can comprise a resilient polymeric material. Suitable polymeric materials for use in flexible circuits are known to one of ordinary skill in the art, e.g., polyamide, polyimide, polyester, or polyethylene terephthalate. In one embodiment, the flexible substrate 20 can have a thickness from about 1 to 10 mils. In some embodiments, the flexible substrate 20 can comprise a material upon which circuitry can be applied through printing, masking or adhesive bonding, for example. In one embodiment, the sensing element 10 can be manufactured using flex circuit technology. Flex circuits have been used in medical devices as microelectrode substrates for in vivo applications. For example, one flex circuit design uses a laminate of a conductive foil (e.g., copper) on a flexible dielectric substrate (e.g., polyamide). The flex circuit can be formed on the conductive foil using masking and photolithography techniques. Flex circuits are desirable due to their small size, low manufacturing cost, ease in design integration, and physical flexibility during transport in applications such as central venous catheter (CVC) insertion. In one embodiment, the invention employs a flex circuit having a length between about 1 and 6 inches, and in particular between about 1 and 3 inches in length. The width of the flex circuit can be from about 0.02 and 0.08 inches, and in particular from about 0.03 to 0.07 inches, and more particularly between about 0.04 to 0.06 inches.

In one embodiment, the adhesive layer 22 comprises a pressure sensitive adhesive that can be used to secure the sensing element 10 within a catheter by contacting the adhesive layer to a surface of the catheter or within a lumen in the catheter. The thickness of the adhesive layer 22 is generally between about 1 to 10 mils, and in particular, between about 2 and 8 mils. Any medical grade pressure sensitive adhesive (PSA) can be used in the adhesive layer 22 including urethane, epoxy, acrylic or silicone PSAs.

The release liner 24 for use in the invention can be any material that adheres to the adhesive layer 24 but that can be easily peeled away from the adhesive layer 24 while keeping the adhesive layer 24 substantially on the substrate 20. The release liner 24 is typically paper, a plastic film, or a combination thereof that can be removed from the adhesive layer 22.

The sensing element 10 can include a wide variety of different sensors that are suitable for measuring a physiological parameter of a patient. For example, the sensing element 10 can include one or more optical fibers, pH sensors, pressure sensors, pacing electrodes, pacing leads, or electrodes for glucose monitoring, and combinations thereof.

In one embodiment, the sensor can comprise one or more electrodes (e.g. 12, 14, 16) mounted on the flexible substrate. For example, in one embodiment, the sensor can comprise an electrochemical sensor in which the occurrence of a chemical reaction can be used to measure/detect a physiological parameter. In one particular embodiment, the sensor can comprise a biosensor, such as an enzyme-based or antibody-based biosensor in which an analyte-concentration-dependent biochemical reaction signal is converted into a measurable physical signal, such as an optical or electrical signal. Such biosensors can be used to measure the concentration of a wide variety of analytes such as glucose, lactate, cholesterol, bilirubin and amino acids. In one particular embodiment, the sensor can comprise a glucose sensor in which an electrode is at least partially coated with a glucose oxidase enzyme. Under proper conditions, when the enzyme electrode is energized and exposed to a flow of blood, oxygen and glucose can react with the enzyme, resulting in an output of electrical current that is proportional to the concentration of glucose in the blood. Excitation of the enzyme electrode and detection of the resulting electrical signal can be achieved by connecting the electrode to external electronics via electrical wires. In addition to sensors for glucose monitoring, other sensors can be used in the invention, such as sensors that measure electrolyte levels in blood or other analytes found in various body fluids.

In one embodiment, the sensing element 10 can comprise an amperometric or potentiometric sensor having one or more electrodes 12, 14 and 16 that can be attached or bonded to a surface of the flexible substrate 20. The sensing element 10 is shown with a reference electrode 12, a separate counter electrode 14, and a working electrode 16. In another embodiment, one or more additional working electrodes can be included on the flexible substrate 20. As noted above, electrical wires 26a, 26b can transmit power to the electrodes for sustaining an oxidation or reduction reaction, and can also carry signal currents to a detection circuit (not shown) indicative of a parameter being measured. The parameter being measured can be any analyte of interest that occurs in, or can be derived from, blood chemistry. In one embodiment, the analyte of interest can be hydrogen peroxide, formed from the reaction of glucose with glucose oxidase, thus having a concentration that is proportional to the blood glucose concentration.

The magnified cross-sectional side view of FIG. 1B shows a distal portion of the substrate 20 in the vicinity of the working electrode 16 taken along line 1B of FIG. 1A. The working electrode 16 can be at least partially coated with a reagent or enzyme layer 28 that is selected to chemically react when the sensor is exposed to certain reactants found in the bloodstream. For example, in an embodiment for a glucose biosensor, enzyme layer 28 can contain glucose oxidase, such as can be derived from Aspergillus niger (EC 1.1.3.4), type II or type VII.

To promote a reaction of the enzyme with blood, the enzyme layer 28 can be formed within a matrix that is active on its surface. This can be achieved, for example, by adding or cross-linking the enzyme to an active hydrogel. The hydrogel layer can be water absorbent, and swell to provide active transport of a reactant in the blood (e.g. glucose) from the blood to the enzyme. Intermolecular bonds can be formed throughout the hydrogel layer to create adhesion and a density of matrix to allow for even dispersion of the reagent across the hydrogel surface and throughout the hydrogel layer. Reaction products can then be communicated to the electrode layer. In some embodiments, the sensing element 10 can also include a flux limiting membrane (not shown) that is added onto the enzyme layer 28 and that at least partially covers the enzyme layer 28. The flux limiting membrane can selectively allow diffusion, from blood to the enzyme layer 28, a blood component that reacts with the enzyme. For example, in a glucose sensor embodiment, the flux limiting membrane passes an abundance of oxygen, and selectively limits glucose, to the enzyme layer 28. In addition, a flux limiting membrane having adhesive properties can be used to mechanically seal the enzyme layer 28 to the working electrode 16, and can also seal the working electrode 16 to the flexible substrate 20. A suitable flux limiting membrane can be formed from an ethylene vinyl acetate (EVA) copolymer, for example. An additional biocompatible layer (not shown), including a biocompatible anti-thrombotic substance such as heparin, can be added onto the flux limiting membrane. Flux limiting membranes are discussed in greater detail in commonly assigned copending patent application Ser. No. 11/710,329, entitled FLUX LIMITING MEMBRANE FOR INTRAVENOUS AMPEROMETRIC BIOSENSOR.

In one embodiment, the sensing element 10 works on an amperometric measurement principle, where the working electrode 16 is held at a positive potential relative to the counter electrode 14. The positive potential is sufficient to sustain an oxidation reaction of hydrogen peroxide, which is the result of a glucose reaction with the glucose oxidase. Thus, the working electrode 16 functions as an anode, and collects electrons produced at the surface of the working electrode 16 that result from the oxidation reaction. The collected electrons flow into the working electrode 16 as an electrical current. When the working electrode 16 is coated with glucose oxidase, the oxidation of glucose produces a hydrogen peroxide molecule for every molecule of glucose, when the working electrode 16 is held at a potential between about +450 mV and about +750 mV. The hydrogen peroxide produced oxidizes at the surface of the working electrode 16 according to the equation:

H₂O₂→2H⁺+O₂+2e⁻

The equation indicates that two electrons are produced for every hydrogen peroxide molecule oxidized. Thus, under certain conditions, the amount of electrical current can be proportional to the hydrogen peroxide concentration. Since one hydrogen peroxide molecule is produced for every glucose molecule oxidized by glucose oxidase, a linear relationship can exist between the blood glucose concentration and the resulting electrical current. The reader can refer to the following article for additional information on electronic sensing theory for amperometric glucose biosensors: J. Wang, “Glucose Biosensors: 40 Years of Advances and Challenges,” Electroanaylsis, Vol. 13, No. 12, pp. 983-988 (2001).

To achieve the linear relationship or substantially linear relationship, the working electrode 16 is designed to promote the desired chemical reactions. In embodiments comprising an amperometric sensor, the chemistry can be controlled by applying one or more membranes, or layers, of varying composition on the surface of the flexible substrate.

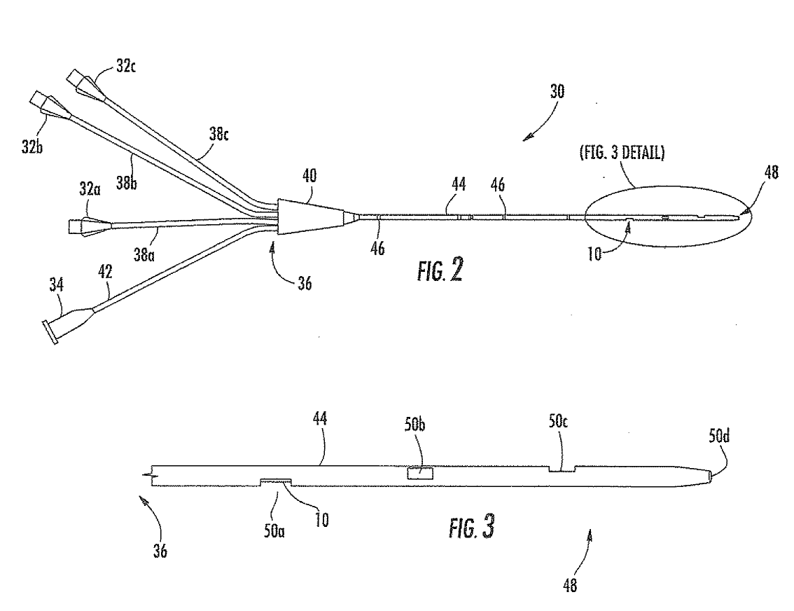

The sensing element can be used in combination with a wide variety of different catheters, such as a single lumen catheter, a multilumen catheter, a central venous catheter (CVC), a pulmonary artery catheter (PAC), or a peripherally inserted central catheter (PICC). In some embodiments, the sensing element can also be used in conjunction with other commonly used peripheral intravenous (IV) lines that provide a suitable platform for effective intravenous positioning of the sensing element. Although the invention can be employed using any of these types of devices, for purposes of illustration only, the invention illustrated in FIGS. 2 and 3 is presented with reference to use with a multilumen CVC. One advantage of using a CVC as a platform for installing an intravenous biosensor can be its ability to reach the largest blood vessels of the body where a biosensor can be exposed to an abundant flow of blood. Further, certain embodiments of the invention can be economically employed for use with multilumen catheters. Thus, the invention is intended to have universal application to catheters.

FIG. 2 illustrates a multilumen catheter assembly 30 in which at least one sensing element 10 (not visible) is integrated into the catheter. The catheter assembly 30 can include multiple infusion ports 32a, 32b, and 32c (collectively referred to as reference number 32) and one or more electrical connectors 34 disposed adjacent to a proximal end 36 of the catheter assembly 30. One or more lumens 38a, 38b, and 38c (collectively referred to as reference number 38) can connect each infusion port 32a, 32b, and 32c, respectively, to a junction 40. Similarly, a conduit 42 can connect an electrical connector 34 to the junction 40, and can terminate at junction 40 or at one of the lumens 38a-38c (as shown). Although the particular embodiment shown in FIG. 2 is a multilumen catheter having three fluid lumens and one electrical lumen, other embodiments having other combinations of lumens and connectors are possible within the scope of the invention, including a single lumen catheter, a catheter having multiple electrical connectors, etc. In another embodiment, one of the lumens and the electrical connector can be reserved for a probe or other sensing element mounting device, or one of the lumens can be open at its proximal end and designated for insertion of the probe or biosensor mounting device.

The junction 40 connects the lumens 38a-38c and the conduit 42 to a narrow elongated tube 44 that forms an insertion portion of the catheter assembly 30. The tube 44 is typically cylindrical, having a circular or somewhat oval cross section defining a longitudinal axis extending therethrough. The tube 44 can be formed from any material, including synthetic materials such as silicone, polyurethane, polyethylene, and the like. Through the junction 40, each of the lumens 38a-38c extend in separate parallel paths for some distance into the distal end of tube 44. One or more support structures 46 within the tube 44 can be disposed along the length of the catheter to provide rigidity.

The distal end 48 of the catheter assembly 30 is shown in greater detail in FIG. 3. At one or more intermediate locations along the distal end, the tube 44 can include one or more recesses formed in an outer wall of the tube. In some embodiments, the recess can define an opening in the outer wall of the tube through which a body fluid can flow into a lumen that is in communication with the opening. In one particular embodiment, the recess can define a port formed in the outer wall of the tube that defines an opening through which a bodily fluid can flow through the port and into the lumen, and vice versa. In the illustrated embodiment, the ports include the intermediate ports 50a, 50b, and 50c, and an end port 50d (collectively referred to as reference number 50) that can be formed towards the distal tip of tube 44. Each port 50a-50d can correspond respectively to one of the lumens 38a-38c or conduit 42. That is, each lumen can define an independent channel extending from one of the infusion ports 32a-32c and conduit 42 to one of the ports 50a-50d located towards the distal end of the tube 44.

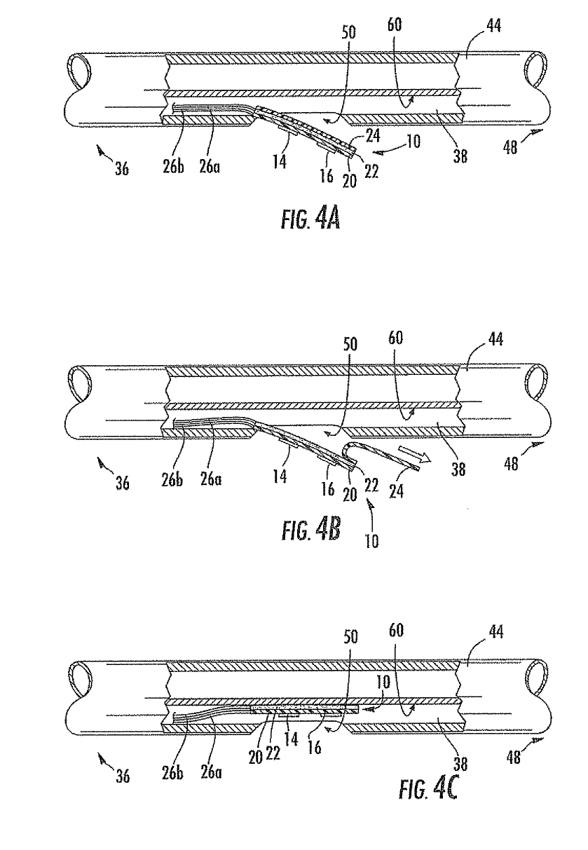

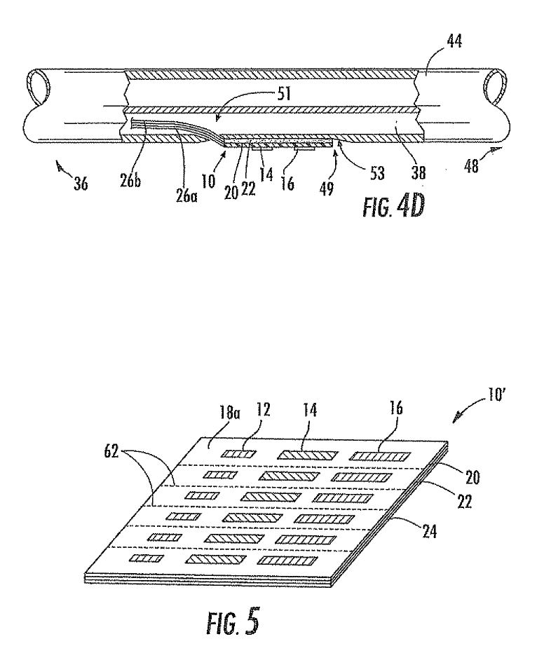

In the embodiment illustrated in FIGS. 4A-4C, port 50a includes a sensing element 10 having a sensor that is exposed for monitoring one or more analytes. A port 50 having a sensing element is referred to herein as a sensing port. In one embodiment, sensing port(s) can perforate an outer wall of catheter assembly 30 to form a hole that opens into a lumen. In one embodiment, the sensing port(s) opens into only one lumen. The sensing port as described herein can be generally oval or rectangular in shape, having a length between about 5.0 and 15.0 mm, and having a width that is generally between about 1.0 mm and about 3.0 mm. The sensing port can be formed in a catheter, for example, by skiving an area of the outer wall of tube 44.

In one embodiment, one or more sensing ports 50 can be located on the tube 44 proximally to an end port, e.g., port 50d. In another embodiment, a catheter can be configured with a single sensing port that is proximal to all other ports, such as port 50a of FIG. 2. In operation within a venous location, the most proximal sensing element (e.g., sensing port 50a) of the catheter can lie advantageously upstream of the distal ports, so that any infusion fluids introduced into the bloodstream through a distal port are prevented from affecting measurements by the sensing element. In applications where it is desirable to position the catheter within an artery, it can be desirable to position the most proximal sensing element (e.g., sensing port) of the catheter upstream of the distal ports, so that any infusion fluids introduced into the bloodstream through a distal port are prevented from affecting measurements by the sensing element.

FIGS. 4A-4C are partial transparent side views of an intermediate portion of the distal end of the catheter of FIG. 2 in which an exemplary method of installing the sensing element in a catheter is illustrated. In the orientation shown, a lumen 38 extends longitudinally within tube 44 along a bottom portion of the catheter. The sensing element 10 can be positioned within the lumen 38 such that its active portion, e.g., the portion containing an electrode, can be exposed to space outside the tube 44 through the port 50. At the proximal end of the sensing element 10, the electrical wires 26a, 26b coupled to electrodes 14, 16 extend from the sensing element through the lumen 38. The electrical wires 26a, 26b are coupled to, or provide, a conductive path through the lumen 38 and the conduit 42 that can terminate at the electrical connector 34. The electrical wires can be attached to the sensor elements with a weld, solder, conductive adhesive, such as a conductive epoxy, and the like. In one embodiment, the electrical wires 26a, 26b can be bonded to the flexible substrate 20 of the sensing element at a proximal location on the substrate. If wire bonding methods are used, the wire attachment region is generally encapsulated with a suitable material, such as a flexible epoxy.

In FIG. 4A, the sensing element 10 is depicted in the process of being inserted into sensing port 50. In one embodiment this step can be accomplished by first inserting and feeding electric wires 26a, 26b through the sensing port 50 and into lumen 38 in the general direction of the proximal end 36 of the catheter assembly. The sensing wires can then be attached to one or more electrical connectors. The presence of the release liner 24 permits the manipulation of the sensing element within the lumen 38 without adversely effecting or disturbing the adhesive layer 22. As a result, the sensing element can be positioned in or adjacent to a desired location within the lumen 38 prior to removing the release liner 24.

As shown in FIG. 4B, once the sensing element 10 is positioned approximate or near a desired location, the release liner 24 can be removed by pulling it away from the adhesive layer 22, to thereby expose the adhesive layer. In FIG. 4C, the sensing element can be attached to the lumen 38 by contacting the exposed adhesive layer to an inner wall 60 of the lumen. In one embodiment, the adhesive layer 22 comprises a pressure sensitive adhesive that permits the sensing element to be securely anchored to an inner wall of the catheter by applying manual pressure against the sensing element 10 in the direction of the inner wall of the catheter, e.g., inner wall 60. The sensing element 10 is generally positioned so that at least a portion of the sensing elements are aligned with sensing port 50.

In one embodiment of the invention, the sensing element 10 can be positioned and secured such that the sensing element is displaced from an inner wall of the catheter or lumen. Positioning the sensing element in a displaced location with respect to the inner wall of the catheter can help reduce the mechanical stress to which the sensing element is exposed during installation, and can also help the sensing element receive an unimpeded and direct flow of blood for sustained measurement accuracy. In this regard, FIG. 4D illustrates an embodiment of the invention in which the sensing element 10 is positioned within recess 49 formed in the outer wall of tube 44. For example, in one embodiment, the recess can be formed by skiving the outer surface of the tube so that a depression is created in which the sensing element can be positioned and attached to surface 53 of the outer wall of tube 44. In some embodiments, the sensing element 10 can be positioned within the recess so that the active portion of the sensing element is flush or nearly flush with the outer surface of the tube. In some embodiments, recess 49 can also include an opening 51 through which electrical wires 26a, 26b can be introduced into the lumen and towards the proximal end 36 of the catheter.

In other embodiments, the sensing element 10 can be connected or mounted inside a length of support tubing (not shown) within the lumen. The support tubing can be formed of material of a desired rigidity similar to the tube 44. In one embodiment, the support tubing can be inserted within the lumen such that it spans the sensing port and positions the active portion of the sensing element facing radially outward and displaced from an inner wall of the catheter (e.g., as shown in FIG. 4D). Additional methods that can be used to position the sensing element within the catheter are discussed in greater detail in commonly assigned copending patent application Ser. No. 11/710,329, entitled CATHETER WITH INTEGRAL BIOSENSOR.

FIG. 5 is a perspective view of a flexible structure 10′ that can be used to prepare a plurality of individual sensing elements that are in accordance with the invention. In one embodiment, the sensing element 10 can be prepared in a process in which one or more electrodes 12, 14, 16 are created on a surface of the flexible substrate 20. As discussed above, the sensing elements can be formed on the flexible substrate 20 using known methods in the art such as masking, printing, adhesive bonding, photolithography techniques, and the like. In one embodiment, multiple rows that each contain one or more sensing elements can be formed on the surface of flexible substrate 20. Thereafter, an adhesive layer 22 can be applied to an opposite surface of the flexible substrate 20. A release liner 24 is then used to cover and protect the adhesive layer 22. In one embodiment, the flexible structure 10′ having multiple sensor elements depicted in FIG. 5 can be divided along lines 62 represented by the dashed lines to form a plurality of individual sensing elements. Electrical wires can be attached to the sensing elements to form a sensing element for use in a catheter as discussed above and are preferably added after the flexible structure 10′ is divided into individual sensing elements.

Many modifications and other embodiments of the invention set forth herein will come to mind to one skilled in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A sensing element for incorporation into a catheter and for monitoring of a physiological parameter in a blood vessel, the sensing element comprising:
a flexible substrate having first and second opposite surfaces;
at least one sensor disposed on the first surface of the flexible substrate;
an adhesive layer covering at least a portion of the second surface of the flexible substrate; and
a release liner releasably adhered to the adhesive layer so that upon removal of the release liner the adhesive layer is exposed for securing the sensing element to the catheter.

2. The sensing element of claim 1, further comprising one or more electrical wires each having an end connected to at least one sensor, and an opposite end for connecting to an electrical connector of the catheter.

3. The sensing element of claim 1, wherein the sensing element comprises a flex circuit.

4. The sensing element of claim 1, wherein the sensor comprises a working electrode and a counter electrode.

5. The sensing element of claim 1, wherein the sensor comprises an electrode having a reactant disposed thereon.

6. The sensing element of claim 5, wherein the reactant comprises glucose oxidase.

7. The sensing element of claim 1, wherein the adhesive layer comprises a pressure sensitive adhesive.

8. The sensing element of claim 1, wherein the adhesive layer covers a substantial portion of the second surface.

9. A catheter assembly for detecting a physiological parameter in a blood vessel, comprising:
an elongated tube having a distal and proximal end, and a longitudinal axis extending therebetween;
a recess formed in an outer wall of the tube between the distal and proximal ends of the tube;
at least one lumen extending longitudinally through the tube and in communication with at least a portion of the recess; and
a sensing element for detecting a physiological parameter in the blood vessel, the sensing element comprising:
a flexible substrate having first and second opposite surfaces;
at least one sensor disposed on the first surface of the flexible substrate;
an adhesive layer covering at least a portion of the second surface of the flexible substrate; and
a release liner releasably adhered to the adhesive layer so that upon removal of the release liner the adhesive layer is exposed for securing the sensing element to the tube adjacent to the recess.

10. The catheter assembly of claim 9, wherein the recess comprises a sensing port formed in the outer wall of the tube and defines an opening through which a bodily fluid can flow through the sensing port and into the lumen.

11. The catheter assembly of claim 9, wherein a plurality of lumens are disposed in the tube.

12. The catheter assembly of claim 9, wherein at least one of the lumens is an infusion lumen having an infusion port for introducing an infusant into the blood vessel.

13. The catheter assembly of claim 12, wherein the infusion port is disposed towards the distal end of the tube with respect to the sensing port.

14. The catheter assembly of claim 9, wherein the sensing element comprises a flex circuit having an amperometric sensor.

15. The catheter assembly of claim 14, wherein the amperometric sensor comprises a working electrode and a reference electrode, and wherein glucose oxidase is disposed on the working electrode for reacting with glucose in the blood vessel.

16. The catheter assembly of claim 15, wherein the amperometric sensor further includes a counter electrode.

17. The catheter assembly of claim 9, wherein the recess includes an opening that is in communication with the lumen through which one or more electrical wires of the sensing element are insertable.

18. A method of preparing a catheter for detecting a physiological parameter in a blood vessel, the method comprising:
providing a catheter assembly having
an elongated tube having a distal and proximal end, and a longitudinal axis extending therebetween,
a recess formed in an outer wall of the tube between the distal and proximal ends of the tube;
positioning a sensing element for detecting the physiological parameter in the blood vessel into the recess, the sensing element comprising:
a flexible substrate having first and second opposite surfaces;
at least one sensor disposed on the first surface of the flexible substrate;
an adhesive layer covering at least a portion of the second surface of the flexible substrate; and
a release liner releasably adhered to the adhesive layer;
removing the release liner to thereby expose the adhesive layer; and
contacting the adhesive layer to a surface of the tube adjacent to the recess to thereby secure the sensing element to the tube.

19. The method of claim 18, wherein the sensing element includes one or more electrical wires each having one end that is connected to at least one sensor and a second end for connecting to an electrical connector of the catheter, the method further comprising the step of feeding the wires into the tube through an opening formed in the recess and towards the proximal end of the catheter.

20. The method of claim 18, wherein the catheter assembly further comprises at least one lumen extending longitudinally through the tube and in communication with the recess, and wherein the recess comprises a sensing port defining an opening through which a bodily fluid can flow through the sensing port and into the lumen.

21. The method of claim 20, further comprising the steps of:
positioning the sensing element within the lumen adjacent to the sensing port; and
contacting the adhesive layer to an inner surface of the lumen adjacent to the sensing port to thereby secure the sensing element to the lumen.

22. The method of claim 18, further comprising the step of forming the sensing element comprising the steps of:
forming a plurality of sensors on the first surface of the flexible substrate;
thereafter, applying a layer of a pressure sensitive adhesive to the second surface of the flexible substrate;
covering the adhesive layer with a release liner; and
separating the flexible substrate into a plurality of individual sensing elements wherein each sensing element includes a working electrode and a counter electrode.

23. The method of claim 18, further comprising the step of applying a reagent to a surface of the sensor.

24. The method of claim 23, wherein the reagent comprises an enzyme.

25. The method of claim 18, wherein the sensing element is positioned in the recess so that it is flush with an outer surface of the tube.

26. The method of claim 18, wherein the adhesive layer of the sensing element covers a substantial portion of the second surface.

27. A method for forming a sensing element for incorporation into a catheter and for monitoring of a physiological parameter in a blood vessel, comprising the steps of:
forming a plurality of sensors on the first surface of the flexible substrate;
thereafter, applying a layer of a pressure sensitive adhesive to the second surface of the flexible substrate;
covering the adhesive layer with a release liner; and
separating the flexible substrate into a plurality of individual sensing elements wherein each sensing element includes a working electrode and a counter electrode.
